(19) United States
(12) Patent Application Publication
(10) Pub. No.: US 2007/0173687 A1
(43) Pub. Date: Jul. 26, 2007

(54) External forceps channel device for endoscope

(76) Inventors: Kiyoteru Shima, Ibaraki (JP); Hideo Kumasaka, Kanagawa (JP)

Correspondence Address: HEDMAN & COSTIGAN P.C., 1185 AVENUE OF THE AMERICAS, NEW YORK, NY, 10036, US

(21) Appl. No.: 10/557,928

(22) PCT Filed: Apr. 23, 2004
(86) PCT No.: PCT/JP04/05854

§ 371 Date: Jan. 17, 2007

(30) Foreign Application Priority Data
May 29, 2003 (JP) 2003-152445

(51) Int. Cl. A61B 1/00

(52) U.S. Cl. 600/106; 600/104; 600/114; 600/128

(57) ABSTRACT

The present invention was made in the light of the foregoing backgrounds, and an object thereof is to provide a forceps channel add-on device for an endoscope, provided on an outer periphery of an insertion portion of the endoscope. The forceps channel add-on device for an endoscope is an external forceps channel device for an endoscope, which is capable of providing two forceps channels or more without controlling the luminous intensity and the field of view of the endoscope, capable of extracting a substance larger than the bore diameter of the forceps channel multiple times while using the endoscope in a state of not being drawn out, without imposing a heavy burden on a patient, and also capable of performing examination after an operation as to whether complications are incurred after the evulsion, whether there is any other affected part overlooked, and so forth. An external forceps channel device for an endoscope, provided with an external forceps channel which is capable of being repeatedly inserted and extracted in a way of being guided by a guide provided on an endoscope separately and independently therefrom along an outside of an insertion portion of the endoscope while using the endoscope without drawing it out, the endoscope incorporating an air supply path, a light source, a CCD camera, and a forceps channel and including the insertion portion and an maneuvering portion, characterized in that provided is the external forceps channel capable of repeatedly extracting a foreign substance larger than a bore diameter of the incorporated forceps channel in a way of being guided by the guide along the outside of the endoscope, together with the whole external forceps channel itself in a state where the foreign substance is grasped by forceps inserted through the external forceps channel, and that provided is the external forceps channel capable of being repeatedly inserted in a way of being guided by the guide along the outside of the endoscope in a state where the endoscope is not drawn out.

### TECHNICAL FIELD

The present invention relates to a forceps channel add-on device for an endoscope and, more particularly, to an external forceps channel device for providing a new forceps channel on an outer periphery of an insertion portion of an endoscope.

### BACKGROUND ART

An existing endoscope normally has one forceps channel, and the forceps channel is not designed to allow the insertion of two pairs of forceps or more. Some endoscopes have two forceps channels. Moreover, among some endoscopes, there exist an endoscope provided with a forceps channel having a bore diameter larger than that of a normal forceps channel to increase the number of usable forceps types and is thereby capable of evulsion of a larger foreign substance; an endoscope provided with a forceps channel on the side to increase a degree of freedom of forceps; and the like. However, to install the forceps channel, the luminous intensity and the field of view of the endoscope had to be controlled.

Moreover, in any case, there was a limitation on the size of a tissue or foreign substance to be extracted. When there were a plurality of tissues and foreign substances which are larger than the bore diameter of the forceps channel, the endoscope had to be inserted again after evulsion. This would impose a heavy burden on a patient. Therefore, when there were a plurality of tissues and the like to be resected which are larger than the bore diameter of a conventional forceps channel, the resected tissues and the like had to be left in the body and separated from feces after examination. When there are a plurality of resected tissues and foreign substances, it is extremely difficult to identify which region each resected tissue or foreign substance was located in.

### Problems to be Solved by the Invention

As described above, in the prior art, there has been no technique for adding a forceps channel which is configured to add a new forceps channel on an outer periphery of an insertion portion of an endoscope and is capable of extracting foreign substances having larger sizes than the bore diameter of the forceps channel many times without drawing out the insertion portion of the endoscope.

However, only one forceps channel may cause many leftovers when performing resection of a tissue, resulting in difficulty in diagnosis. Moreover, an existing endoscope alone cannot evulse a substance larger than the bore diameter of the forceps channel without drawing out the endoscope itself. Moreover, in the case of the existing endoscope, when evulsing a substance larger than the bore diameter of the forceps channel, there is no other method but to repeat the extraction and insertion of the insertion portion of the endoscope every single time. When substances to be evulsed are numerous such as multiple polyps, a patient and an operator bear enormous burdens.

Accordingly, upon resection of the multiple polyps and the like larger than the bore diameter of the forceps channel with an existing endoscope, when it is difficult to repeat the extraction and insertion of the insertion portion of the endoscope every single time in consideration for burdens on a patient and an operator, the resected tissues are left in the body and separated from feces after examination. This imposes a great burden on a medical personnel side. Moreover, it is difficult to identify which region the resected tissue thus separated comes from, resulting in degradation in regional diagnostic capability of endoscopy. Moreover, there are some occasions when an endoscope needs to be drawn out after a treatment with the endoscope in order to evulse a tissue larger than the bore diameter of the forceps channel or the like. In such a case, there is an increasing risk of overlooking complications such as bleeding, and a burden on a patient is increased, for example, when the endoscope needs to be inserted again.

Furthermore, since the forceps channel also serves as a suction port during suction, suction capability of the endoscope is reduced when forceps are inserted. Moreover, when the field of view of the endoscope is bad due to bleeding or the like, forceps are inserted for a treatment to stop bleeding and the like. Here, suction power may be weakened by such insertion and the field of view of the endoscope may become worse, endangering the life of a patient and the like.

The present invention was made in the light of the foregoing backgrounds, and an object thereof is to provide a forceps channel add-on device for an endoscope, provided on an outer periphery of an insertion portion of the endoscope. The forceps channel add-on device for an endoscope is an external forceps channel device for an endoscope, which is capable of providing two forceps channels or more without controlling the luminous intensity and the field of view of the endoscope, capable of extracting a substance larger than the bore diameter of the forceps channel multiple times while using the endoscope in a state of not being drawn out, without imposing a heavy burden on a patient, and also capable of performing examination after an operation as to whether complications are incurred after the evulsion, whether there is any other affected part overlooked, and so forth.

### Means for Solving the Problems

An external forceps channel device for an endoscope of the present invention is provided with an external forceps channel which is capable of being repeatedly inserted and extracted in a way of being guided by a guide provided on an endoscope separately and independently therefrom, along an outside of an insertion portion of the endoscope, while using the endoscope without drawing it out, the endoscope incorporating an air supply path, a light source, a CCD camera, and a forceps channel and including the insertion portion and an maneuvering portion. The external forceps channel device for an endoscope is characterized in that provided is the external forceps channel capable of repeatedly extracting a foreign substance larger than a bore diameter of the incorporated forceps channel in a way of being guided by the guide along the outside of the endoscope, together with the whole external forceps channel itself, in a state where the foreign substance is grasped by forceps inserted through the external forceps channel, and that provided is the external forceps channel capable of being repeatedly inserted in a way of being guided by the guide along the outside of the endoscope in a state where the endoscope is not drawn out.

The external forceps channel device for an endoscope of the present invention set forth in claim 1 is an external forceps channel device for an endoscope capable of being slidably fitted along the outside of the insertion portion of an existing endoscope, and is characterized in that detachable notched ring-shaped fitting pieces are provided on an outer peripheral surface of the insertion portion at given intervals; a linear member having flexibility and elasticity is fixed to one end side of the fitting pieces to interconnect the fitting pieces; a tube having flexibility with a cross-section of a C-shape is fixed to the other end side of the fitting pieces in parallel with the linear member to interconnect the fitting pieces; and the external forceps channel connected with a rod guide portion that is slidably insertable into and detachable from the C-shaped-cross-section tube is provided in the C-shaped-cross-section tube.

The external forceps channel device for an endoscope of the present invention set forth in claim 2 is characterized in that the linear member is any of a tension coil spring with no hook on either end and a rod member made of thermoplastic resin having flexibility and elasticity such as a nylon material.

The external forceps channel device for an endoscope of the present invention set forth in claim 2 or 3 is characterized in that a guide ring fitted on a tip of the external forceps channel device for an endoscope is formed into a shape having a protruded center, a guide portion is formed by cutting the guide ring obliquely from a tip toward a base portion thereof, and a positioning cap formed to match with the shape of the guide portion is fitted on a tip of the insertion portion of the endoscope, and that, with the above structure, the guide ring on the tip of the external forceps channel device for an endoscope set forth in claim 2 or 3 is positioned by aligning the guide ring on the tip of the device with the positioning cap fitted on the tip of the endoscope, at the tip of the endoscope when the external forceps channel device for an endoscope ser forth in claim 2 or 3 is inserted.

The external forceps channel device for an endoscope of the present invention set forth in claim 1 is characterized in that the external forceps channel is provided either in such a manner that a groove having a cross-section of a C-shape is provided along a surface of the insertion portion of the endoscope in its longitudinal direction, and the external forceps channel connected with a rod guide portion which is insertable into and detachable from the C-shaped-cross-section groove while sliding therealong, is provided, or in such a manner that a protruding guide member is provided along the surface of the insertion portion of the endoscope in its longitudinal direction, and the external forceps channel connected with the rod guide portion which is insertable into and detachable from the guide member while being guided by the guide member, is provided.

The external forceps channel device of an endoscope is characterized in that the external forceps channel for the endoscope of the present invention set forth in claim 5 is an external forceps channel one side of which is connected with the rod guide portion that is insertable into any of the C-shaped-cross-section groove and the protruding guide member and slidably insertable into and detachable from the cross-section of any of the C-shaped-cross-section groove and the protruding guide member, and that a guide wire as a core is provided in a central portion of the rod guide portion.

The external forceps channel device for an endoscope is characterized in that, for the external forceps channel for the endoscope set forth in claim 5 or 6, an open portion of any of the C-shaped-cross-section groove and the protruding guide member is formed to have an elastic constrictive structure to prevent the external forceps channel from protruding out, the external forceps channel being connected with the rod guide portion slidably inserted into any of the C-shaped-cross-section groove and the protruding guide member.

The external forceps channel device for an endoscope of the present invention set forth in claim 1 is characterized by adopting a structure in which a tunnel is provided immediately under a surface of the insertion portion of the endoscope along the surface in its longitudinal direction; a freely movable magnetic body is provided in the tunnel; another magnetic body is also provided at a bottom portion of a tube through which forceps are inserted along the outside of the insertion portion of the endoscope; and when inserting the external forceps channel device, the device is inserted while being guided by the movable magnetic body.

The external forceps channel device for an endoscope is characterized in that, for the external forceps channel for the endoscope of the present invention set forth in claim 5 or 6, a partition plate is provided on a tip of the flexible rod guide portion which is slidably insertable into and detachable from any of the C-shaped-cross-section groove and the protruding guide member; the partition plate is pushed forward by air pressure; and the external forceps channel is connected with the rod guide portion.

### BRIEF DESCRIPTION OF THE DRAWINGS

FIG. 1 is a side view of an external forceps channel device for an endoscope, representing one embodiment of the present invention, showing a case of being added to an existing endoscope.

FIG. 2 is a plan view of the external forceps channel device for an endoscope, representing the one embodiment of the present invention, showing the case of being added to the existing endoscope.

FIG. 3 is a front view of the external forceps channel device for an endoscope, representing the one embodiment of the present invention, showing the case of being added to the existing endoscope.

FIG. 4 is a cross-sectional view, taken along the A-A′ line in FIG. 2, of the external forceps channel device for an endoscope, representing the one embodiment of the present invention, showing the case of being added to the existing endoscope.

FIG. 5 is a perspective view of an external forceps channel connected with a rod guide portion.

FIG. 6 is a cross-sectional view of the external forceps channel connected with the rod guide portion, taken along the B-B′ line in FIG. 5.

FIG. 7 is a cross-sectional view, taken along the A-A′ line in FIG. 2, of the external forceps channel device for an endoscope, representing the one embodiment of the present invention, in a state where the rod guide portion connected with the external forceps channel is inserted into a C-shaped-cross-section tube of the external forceps channel device for an endoscope, showing the case of being added to the existing endoscope.

FIG. 8 is a side view of a forceps channel add-on device for an endoscope, representing another embodiment of the present invention in a case of having an integrated structure, showing the case where a C-shaped-cross-section groove is provided along a surface of the insertion portion of the endoscope in its longitudinal direction, and further, a guide wire is passed through the rod guide portion connected with the external forceps channel.

FIG. 9 is a cross-sectional view of the forceps channel add-on device for an endoscope, representing the other embodiment of the present invention in the case of having the integrated structure, taken along the C-C′ line in FIG. 8.

FIG. 10 is a side view of a forceps channel add-on device for an endoscope, representing another embodiment of the present invention in a case of having an integrated structure, showing the case where a protruding guide member is provided along the surface of the insertion portion of the endoscope in its longitudinal direction, and further, the guide wire is passed through the rod guide portion connected with the external forceps channel.

FIG. 11 is a cross-sectional view of the forceps channel add-on device for an endoscope, representing the other embodiment of the present invention in the case of having the integrated structure, taken along the D-D′ line in FIG. 10.

FIG. 12 is a side view of a forceps channel add-on device for an endoscope, representing another embodiment of the present invention in a case of having an integrated structure, showing the case where an open portion with a C-shaped cross-section is formed to have an elastic constrictive structure.

FIG. 13 is a cross-sectional view of the forceps channel add-on device for an endoscope, representing the other embodiment of the present invention in the case of having the integrated structure, taken along the E-E′ line in FIG. 12.

FIG. 14 is a side view of a forceps channel add-on device for an endoscope, representing another embodiment of the present invention in a case of having an integrated structure, showing the case where a tunnel is provided immediately under an outer peripheral surface of the insertion portion of the endoscope; freely movable magnetic bodies are provided in the said tunnel; and another magnetic bodies are also provided at a bottom portion of the external forceps channel.

FIG. 15 is a cross-sectional view of the forceps channel add-on device for an endoscope, representing the other embodiment of the present invention in the case of having the integrated structure, taken along the F-F′ line in FIG. 14.

FIG. 16 is a side view of a forceps channel add-on device for an endoscope, representing another embodiment of the present invention in a case of having an integrated structure, showing the case where a cross-section of a guide portion connected with the external forceps channel is formed into a C shape; and a rail which the said C-shaped-cross-section guide portion is engaged with, insertable into, detachable from, and smoothly movable along, is provided on a C-shaped-cross-section groove or a guide member.

FIG. 17 is a cross-sectional view of the forceps channel add-on device for an endoscope, representing the other embodiment of the present invention in the case of having the integrated structure, taken along the G-G′ line in FIG. 16.

FIG. 18 is photographic representations of perspective views showing an overall image and an operating portion of a conventional colon endoscope.

FIG. 19 is a photographic representation of a front view showing a tip of the conventional colon endoscope and a photographic representation of a schematic diagram showing a state of the tip provided with an air supply port, a light source, a camera, and a forceps channel (about 3 mm in diameter).

FIG. 20 is photographic representations of perspective views showing a method of inserting forceps into the conventional colon endoscope, showing insertion of the forceps from the operating portion and the forceps coming out of the tip of the endoscope.

FIG. 21 is a photographic representation of a conventional method of endoscopically resecting a colon polyp.

FIG. 22 is photographic representations of an overall image of a colon and a case of a descending colon polyp.

FIG. 23 is endoscopic photographs showing a small polyp removable through a forceps channel and a polyp far larger than the diameter of the forceps channel.

FIG. 24 is endoscopic photographs showing resection of the small polyp.

FIG. 25 is endoscopic photographs showing resection of the large polyp.

FIG. 26 is endoscopic photographs showing conventional removal of a large polyp.

FIG. 27 is photographic representations of an overall image and an enlarged image of a tip portion showing one example of the present invention.

FIG. 28 is photographic representations of a C-shaped tube and a spectacle-shaped tube used as the external forceps channel connected with the rod guide, showing one example of the present invention.

FIG. 29 is a photograph showing a state of inserting the spectacle-shaped tube into the C-shaped tube, showing the one example of the present invention.

FIG. 30 is photographs showing a tip portion of an external forceps channel device for an endoscope and an enlarged image thereof, showing one example of the present invention.

FIG. 31 is photographs showing a positioning cap, showing one example of the present invention.

FIG. 32 is photographs showing movement of a tip of an external forceps channel by the positioning cap, showing the one example of the present invention.

FIG. 33 is a photograph representing an operation procedure 1, showing one example of the present invention.

FIG. 34 is a photograph representing an operation procedure 2, showing the one example of the present invention.

FIG. 35 is a photograph representing an operation procedure 3, showing the one example of the present invention.

FIG. 36 is a photograph representing an operation procedure 4, showing the one example of the present invention.

FIG. 37 is photographs showing the use of a training device.

FIG. 38 is photographs showing the use of the training device, showing a state of insertion of an endoscope.

FIG. 39 is photographs showing a state of insertion of an external forceps channel device according to the present invention by use of the training device.

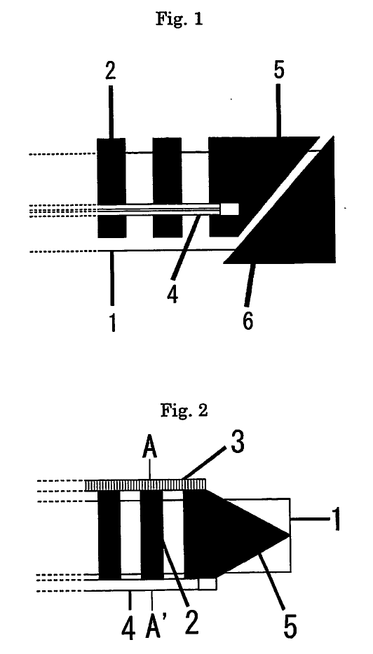

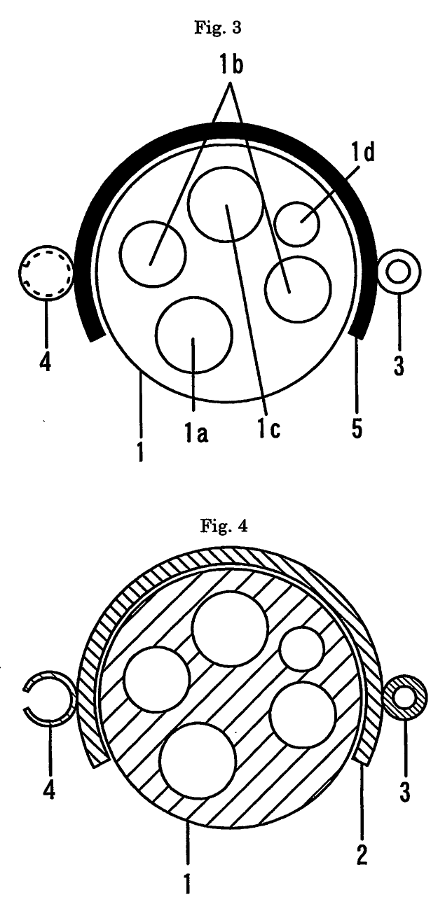

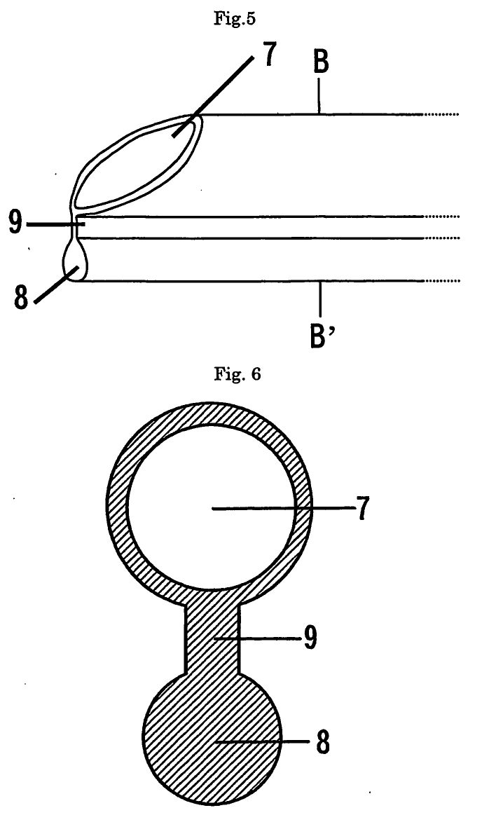

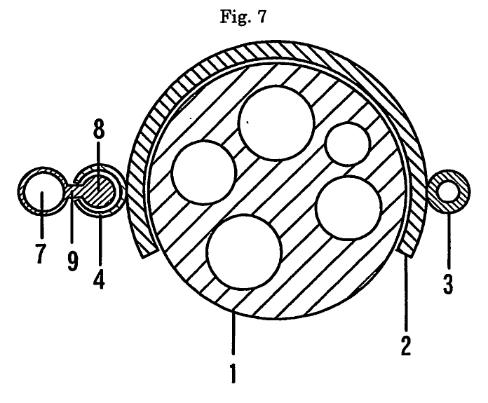

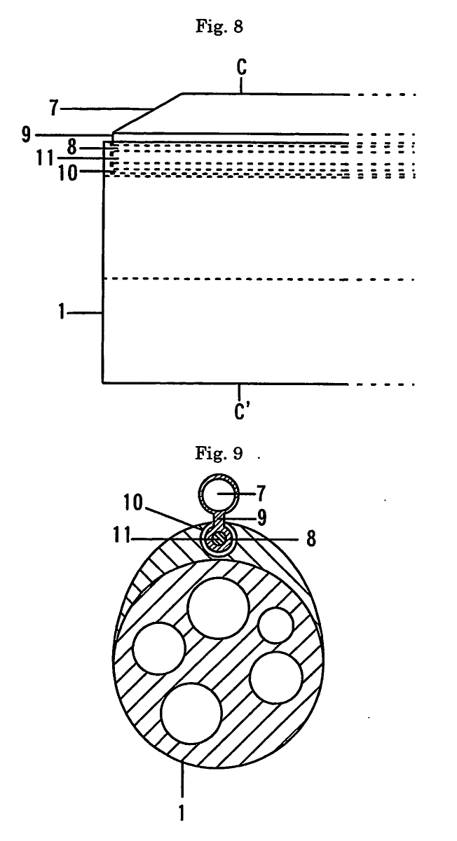

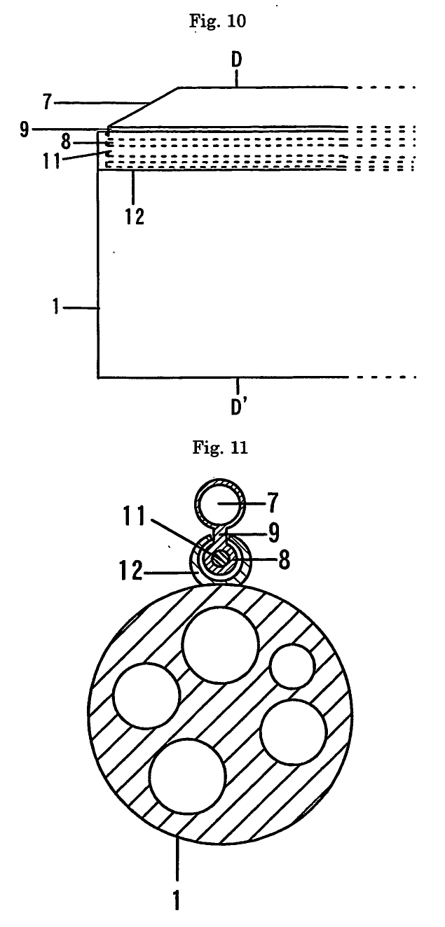

Reference numeral 1 denotes an insertion portion of an endoscope; 1a, a forceps channel; 1b, a light source; 1c, a CCD camera; 1d, an air supply hole; 2, a ring-like fitting piece; 3, a linear member; 4, a C-shaped tube; 5, a tip guide ring; 6, a positioning cap; 7, an external forceps channel; 8, a rod guide portion; 9, a connecting portion between the external forceps channel and the rod guide portion; 10, a C-shaped-cross-section groove; 11, a guide wire; 12, a guide member protruding along a surface of the insertion portion of the endoscope in its longitudinal direction; 13, a material having elasticity; 14, a tunnel; 15a, a magnetic body (inside the tunnel); 15b, a magnetic body (at a bottom portion of the external forceps channel); 16, a wire connecting a plurality of magnetic bodies; 17, a C-shaped-cross-section groove of the rod guide portion; and 18, a rail.

### MODES FOR CARRYING OUT THE INVENTION

Hereinafter, some of embodiments of the present invention will be described in detail based on the drawings.

The present invention forms an external forceps channel device which is capable of sidably fitting along an outside of an insertion portion (1) of an existing endoscope incorporating an air supply path (1d), a light source (1b), a CCD camera (1c), and a forceps channel (1a) and also including an insertion portion and a maneuvering portion.

When providing the external forceps channel device for an endoscope on an outer periphery of the insertion portion (1) of the existing endoscope, detachable ring-like fitting pieces (2) made of, for example, plastic synthetic resin which is harmless when used in a human body are provided on an outer peripheral surface of the insertion portion (1) at given intervals. A linear member (3) having flexibility and elasticity, for example, a tension coil spring having a diameter of about 1 mm with no hooks, is fixed to one end side of the fitting pieces (2). Meanwhile, a material having flexibility, for example, a tube (4) having a cross-section of a C-shape made of silicon resin, is fixed to the other end side of the fitting pieces in parallel with the linear member (3), with its cut side oriented outward at the opposite position to the linear member (3).

Here, elasticity means an ability of fitting in a deformed condition of the insertion portion and not bending even if force is applied in a longitudinal direction upon insertion.

Moreover, the C-shaped-cross-section tube (4) is provided with an external forceps channel (7) connected with a rod guide portion (8) which is slidably insertable into and detachable from the C-shaped-cross-section tube (4).

A spectacle-shaped tube which is made of, for example, silicon resin that is harmless even when used in a human body has is used as the external forceps channel (7). One side of the spectacle-shaped tube is in a rod-shape to be insertable into the C-shaped-cross-section tube (4), and the other side is in a pipe-shape with a size capable of allowing insertion of forceps. The spectacle-shaped tube includes the external forceps channel (7) through which forceps are inserted, a rod guide portion (8) annexed to the external forceps channel, and a connecting portion (9) between the external forceps channel and the rod guide portion. A cross section of an endoscope-tip reaching portion of the external forceps channel in the spectacle-shaped tube is made oblique outwards relative to the outer periphery of the insertion portion of the endoscope.

A tip guide ring (5) is provided at a tip of the above-described external forceps channel device for an endoscope of a type to be added to an existing endoscope. The tip guide ring (5) is made of, for example, plastic synthetic resin which is harmless when used in a human body and the tip guide ring (5) is formed into an obliquely cut shape from a tip toward a base portion thereof. In addition, a positioning cap (6) which matches with the shape of the tip guide ring (5) is fitted and fixed onto a tip of the endoscope in advance.

On the other hand, as an embodiment of providing the external forceps channel device for an endoscope on the outer periphery at the manufacturing stage of an endoscope, a C-shaped-cross-section groove (10), into/from which the rod guide portion (8) connected with the external forceps channel (7) like the spectacle-shaped tube, for example, can be slidably inserted and detached, is provided along the surface of the insertion portion of the endoscope in its longitudinal direction. Alternatively, a guide member (12) protruding along the surface of the insertion portion of the endoscope in its longitudinal direction is provided.

A linear member having flexibility and elasticity, for example, a guide wire (11), is passed in advance through the rod guide portion of the external forceps channel (7) which includes the rod guide portion (8) that is insertable into and detachable from either the C-shaped-cross-section groove (10) or the protruding guide member (12), for example, the rod guide portion of the aforementioned spectacle-shaped tube.

As another embodiment of providing the external forceps channel device for an endoscope on the outer periphery at the manufacturing stage of the endoscope, an open portion of either the C-shaped-cross-section groove (10) or the protruding guide member (12) is formed to have an elastic constrictive structure. For example, both sides or one side of the open portion of either the C-shaped-cross-section groove (10) or the protruding guide member (12) is formed of a material (13) having elasticity such as rubber; the structure adopts a closed structure when the rod guide portion (8) connected with the external forceps channel (7) is not inserted in the C-shaped-cross-section groove (10); and the structure adopts a structure to pressurize the connecting portion (9) between the external forceps channel (7) and the rod guide portion when the rod guide portion (8) is inserted in either the C-shaped-cross-section groove (10) or the protruding guide member (12).

As still another embodiment of providing the external forceps channel device for an endoscope on the outer periphery at the manufacturing stage of the endoscope, a tunnel (14) is provided in an outer peripheral edge of the insertion portion of the endoscope, and inside the tunnel, provided are a plurality of magnetic bodies (15a), for example, permanent magnets, which are freely movable along the surface of the insertion portion of the endoscope in its longitudinal direction. The plurality of magnetic bodies are connected to each other by a wire (16) or the like. Meanwhile, a plurality of magnetic bodies (15b), for example, permanent magnets, are also provided at a bottom portion of a tube constituting the external forceps channel (7) to be inserted along the outside of the insertion portion of the endoscope. The magnetic bodies (15a) at the bottom portion of the tip of the tube of the external forceps channel and the magnetic bodies (15b) inside the tunnel (15b) attract one another while sandwiching the outer peripheral surface of the insertion portion of the endoscope, thus adopting a structure in which the external forceps channel (7) travels along the outer peripheral surface of the insertion portion of the endoscope while being guided by the movable magnetic bodies.

It is preferable that the external forceps channel (7), for example, the tube having a size capable of allowing insertion of forceps, adopt a structure which allows an increased area of the attraction in the outer peripheral surface of the insertion portion of the endoscope.

As yet another embodiment of providing the external forceps channel device for an endoscope on the outer periphery at the manufacturing stage of the endoscope, a cross section of the rod guide portion (8) connected with the external forceps channel (7) is formed into a C-shape to provide a groove (17), and a rail (18) which the C-shaped-cross-section guide portion is engaged with, is insertable into and detachable from, and is smoothly movable along, is provided on either the C-shaped-cross-section groove (10) provided along the surface of the insertion portion of the endoscope in its longitudinal direction or the protruding guide member (12) provided along the surface of the insertion portion of the endoscope in its longitudinal direction.

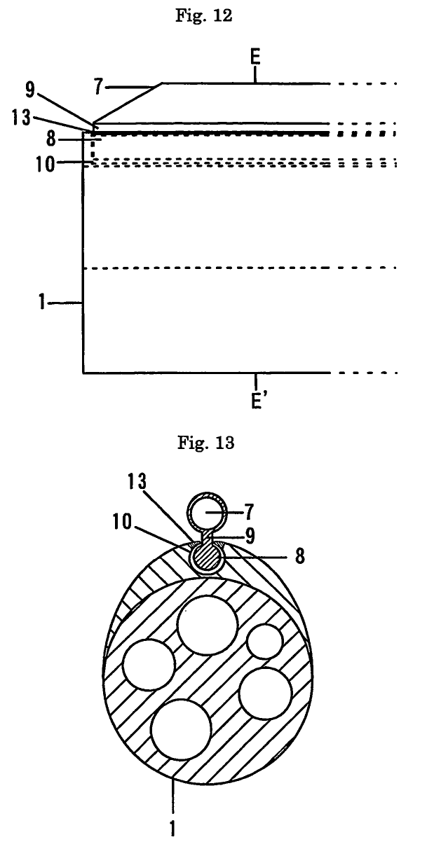

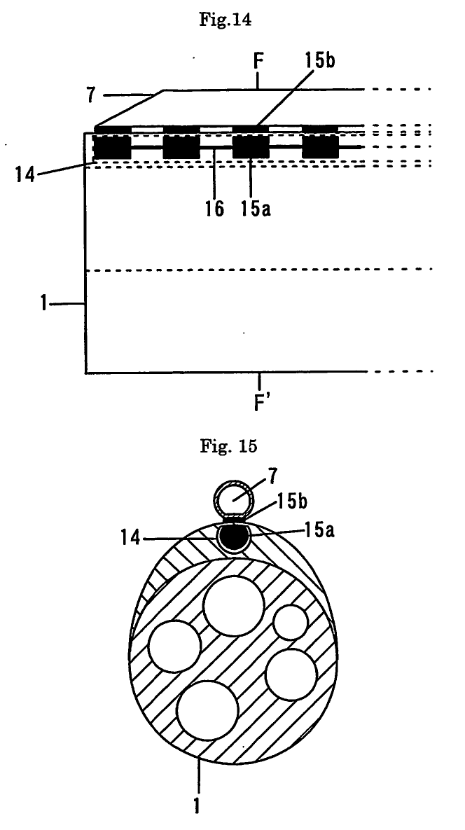

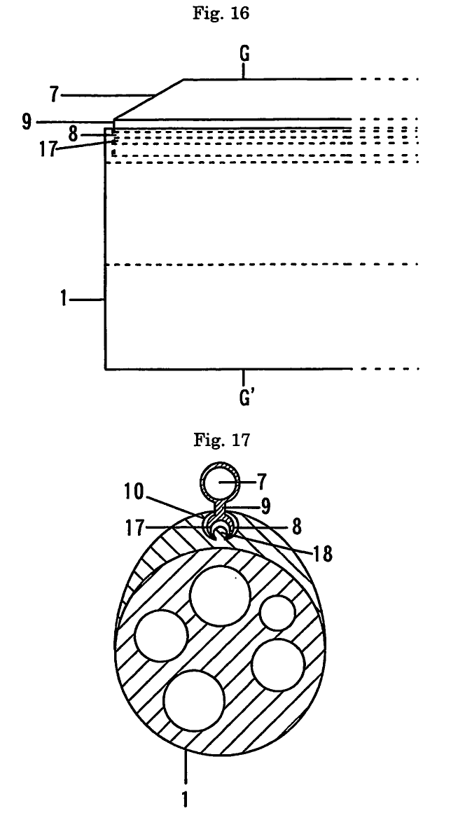

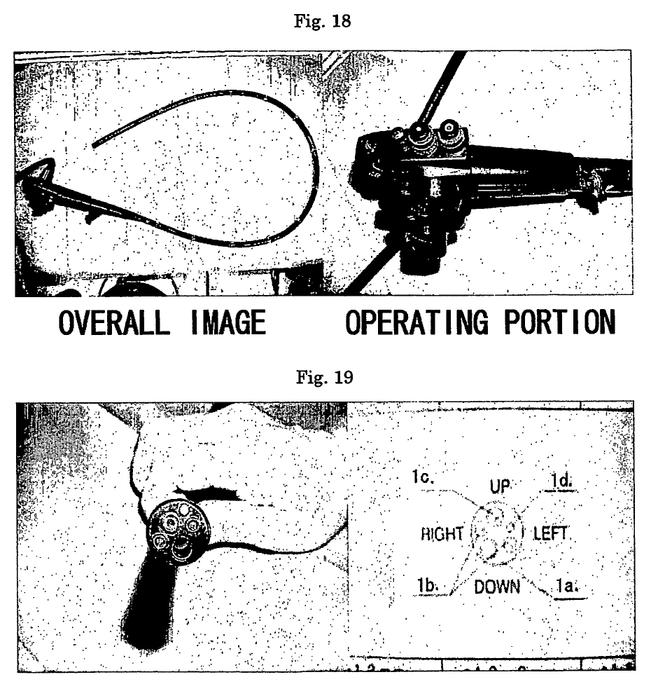

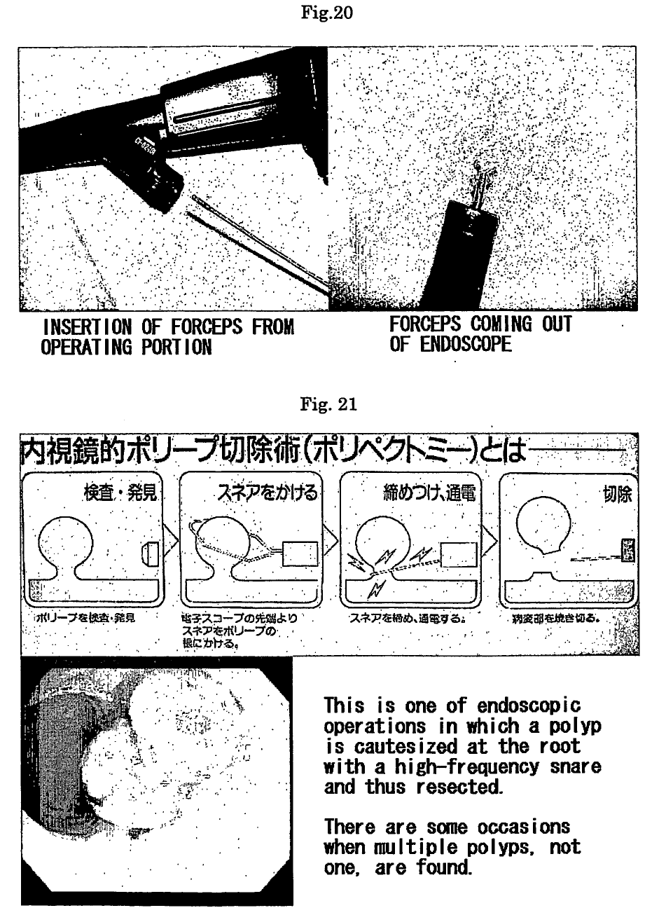

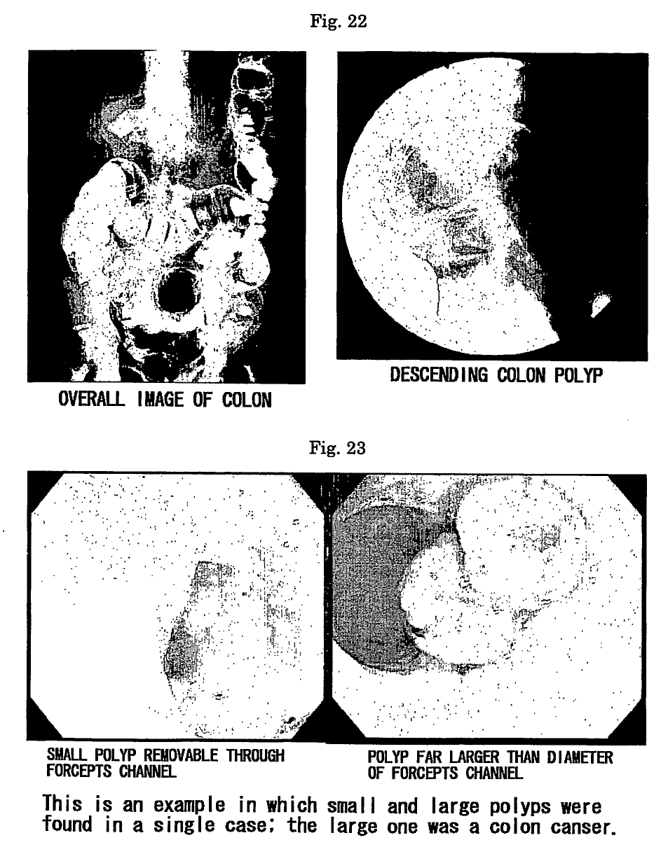

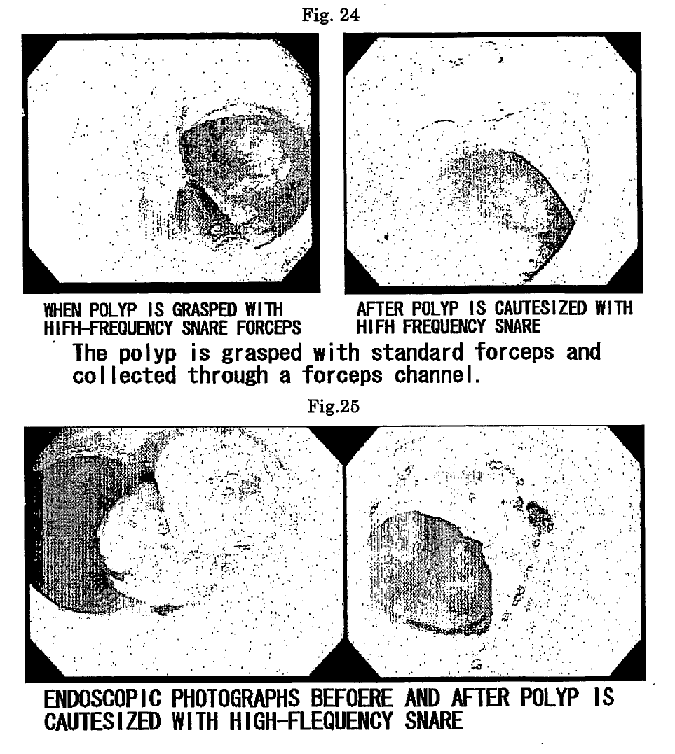

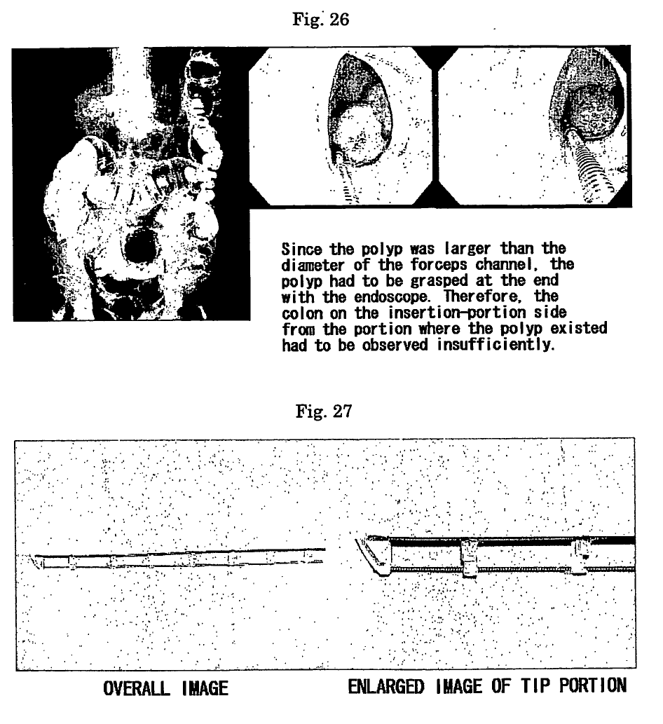

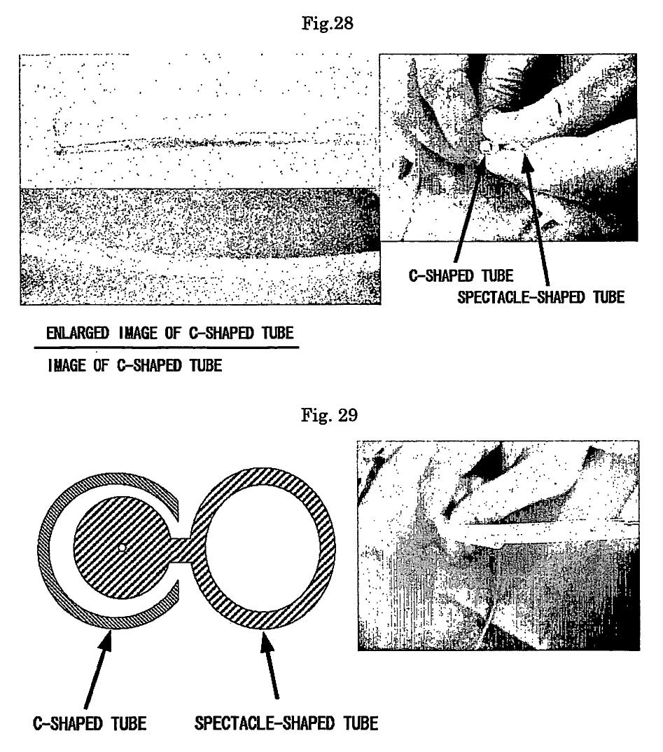

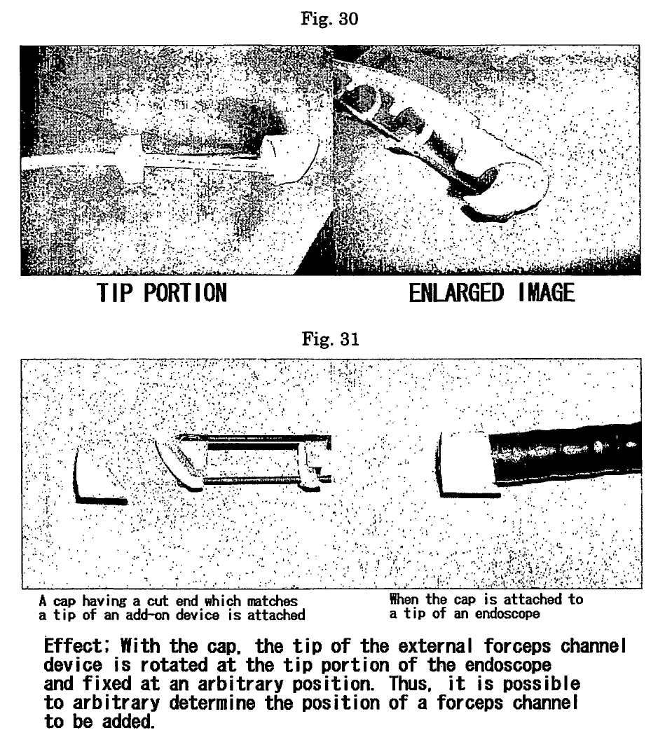

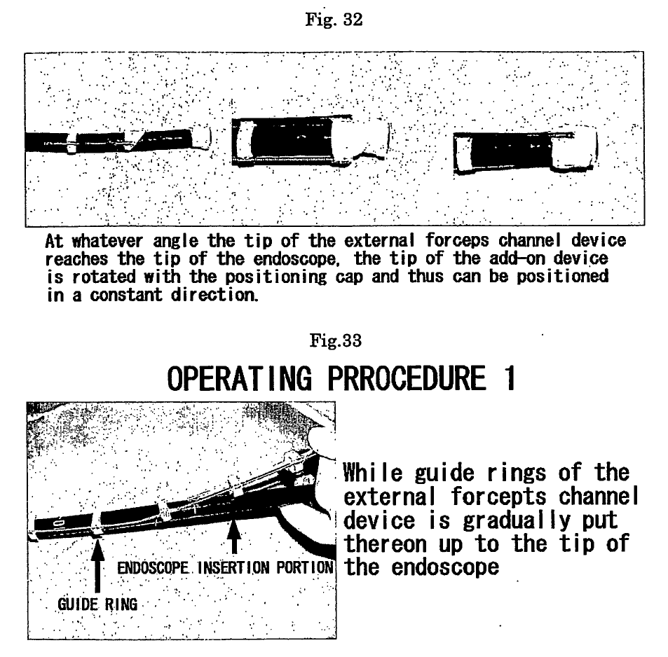

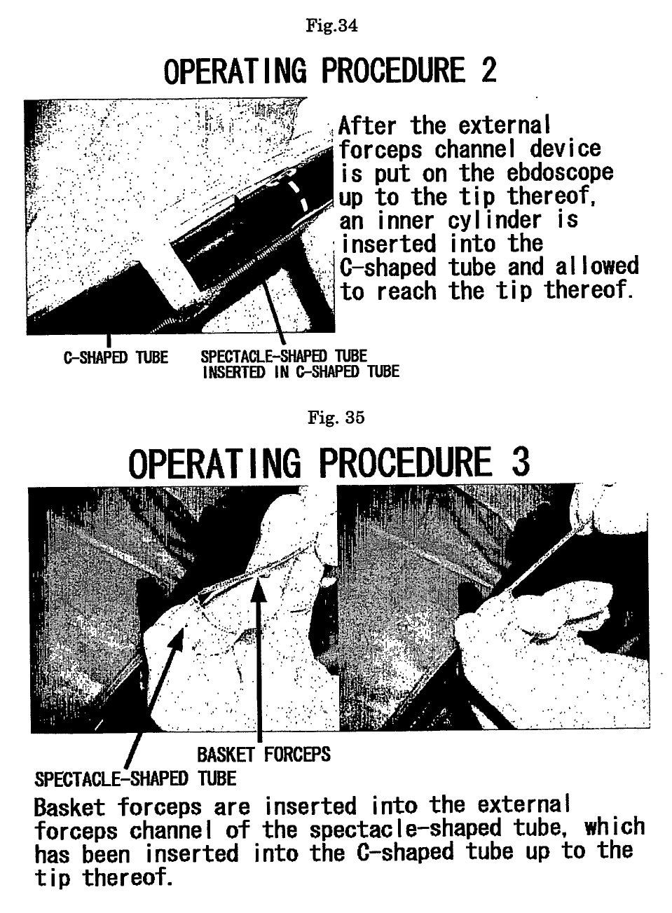

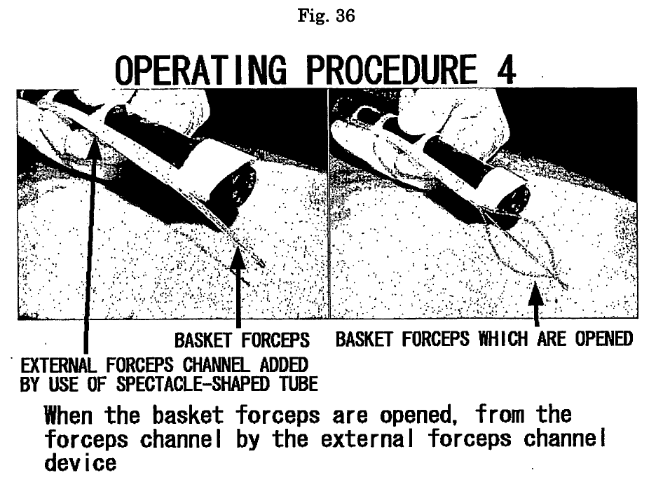

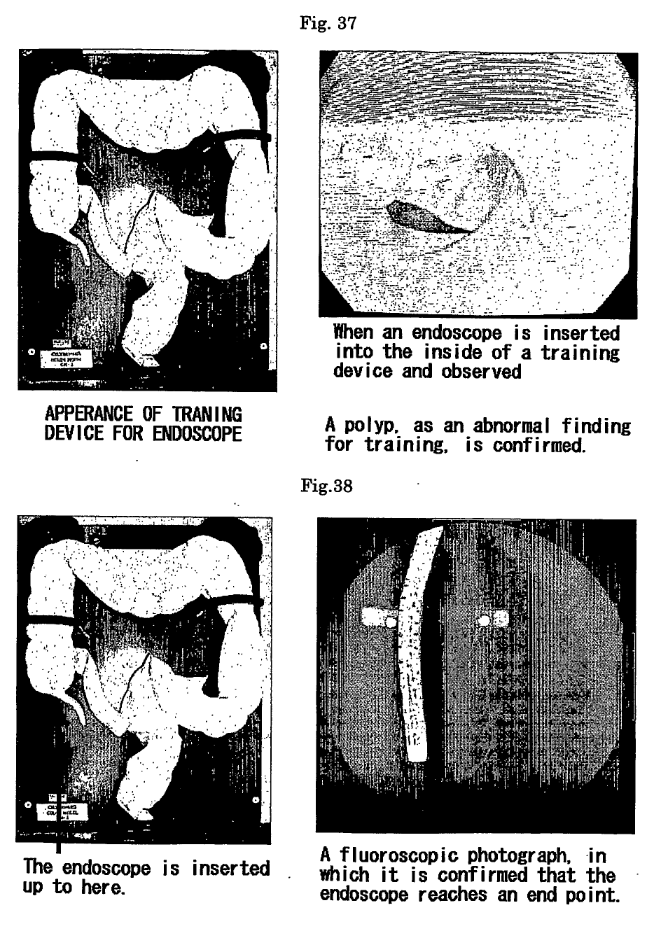

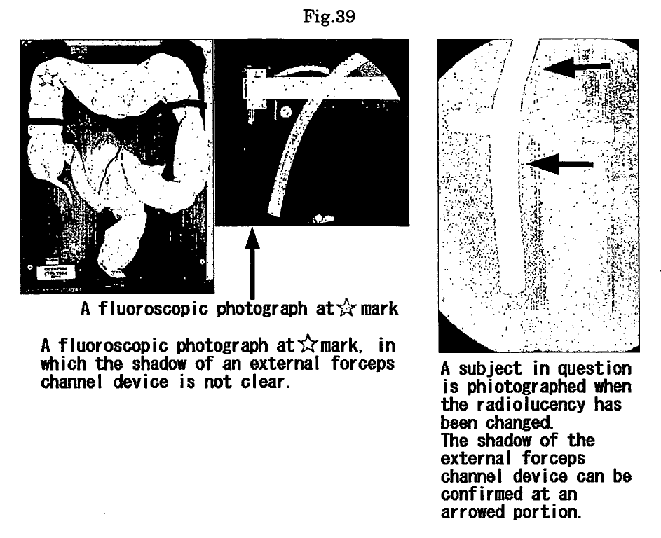

In the embodiments of the present invention, the peripheral edge of the C-shaped-cross-section groove in each of FIGS. 9, 13, and 17 has been described as a different material from that of a surface layer of the insertion portion of the endoscope. However, it is possible to integrally form the peripheral edge by use of the same material. The same is true of the protruding guide member in FIG. 11 and of a peripheral edge of the tunnel in FIG. 15.

Moreover, FIGS. 9, 13, and 17 each show a structure in which the rod guide portion connected with the external forceps channel is inserted into and detached from the C-shaped-cross-section groove provided along the surface of the insertion portion of the endoscope in its longitudinal direction. However, similarly to FIG. 11, it is also possible to use a guide member which protrudes along the surface of the insertion portion of the endoscope in its longitudinal direction.

### EXAMPLE

As an example of the present invention, description will be given of the external forceps channel device for an endoscope, to be provided on the outer periphery of the insertion portion of an existing endoscope, and of a use example thereof in a colon camera as an example of the endoscope.

Procedures for Use

Conditions of a case to which the use of the present invention is applicable include absence of high-degree adhesions after an operation or the like, and implementability under radioscopy.

The following works are performed as preparation prior to the implementation of the present invention.

A sufficient quantity of a lubricant is applied to the colon camera. To facilitate insertion of the colon camera and the external forceps channel device for an endoscope, a dilator or a slider is used as a device for dilating an anal region. When using the external forceps channel device for an endoscope to be annexed to an existing endoscope, the positioning cap for positioning is firmly fitted on and fixed to the tip of the endoscope.

Use procedures are as follows.

A sufficient quantity of a lubricant is applied to a region for insertion of a patient's body.

Next, when using the slider, the slider is fitted on the endoscope. When using the dilator on an anus, the dilator is inserted into the anal region and a tip thereof is opened to dilate the anus.

Subsequently, the colon camera fitted with the slider is inserted into the dilated anus.

When using the external forceps channel device for an endoscope of the present invention which is to be annexed to the existing endoscope, the colon camera is allowed to reach a target region, and thereafter the external forceps channel device for an endoscope is fitted onto a main body of the colon camera and is subsequently inserted. In this event, a sufficient quantity of a lubricant is applied to the external forceps channel device for an endoscope.

The external forceps channel device for an endoscope is inserted while seeing inside the body by radioscopy. The tip portion of the endoscope including the colon camera is an adjustable portion which enables the endoscope to be freely oriented. The external forceps channel device for an endoscope is allowed to reach close to the adjustable portion.

When the external forceps channel device for an endoscope reaches close to the adjustable portion, the adjustable portion is manipulated to be as straight as possible while observing it by radioscopy.

When the adjustable portion is made straight, the external forceps channel device for an endoscope is further inserted slowly until the tip guide ring thereof abuts on the positioning cap fixed to the tip of the colon camera and is rolled and fixed thereto.

After inserting the external forceps channel device for an endoscope to the tip of the colon camera, the adjustable portion of the endoscope is manipulated again to confirm the target.

After the confirmation of the target, the rod guide portion connected with the external forceps channel, for example, the rod guide portion of the spectacle-shaped tube, is inserted to the C-shaped-cross-section tube of the external forceps channel device for an endoscope. In this event, a sufficient quantity of a lubricant is applied to the spectacle-shaped tube, and at the same time the guide wire is passed through a forceps channel insertion portion of the spectacle-shaped tube.

The spectacle-shaped tube is slowly inserted to the C-shaped-cross-section tube until the spectacle-shaped tube cannot be inserted any further, thereby allowing the spectacle-shaped tube to reach the tip of the external forceps channel device.

The guide wire is drawn out of the forceps channel insertion portion of the spectacle-shaped tube, and forceps considered as necessary is inserted into the forceps channel insertion portion of the spectacle-shaped tube and then used.

When removing a foreign substance in the body by using the forceps inserted through the forceps channel insertion portion of the spectacle-shaped tube, for example, by using basket forceps, the foreign substance is remover by extracting the forceps together with the spectacle-shaped tube if it is impossible to take the foreign substance out through the forceps channel of the main body of the existing endoscope or through the forceps channel insertion portion of the spectacle-shaped tube.

Subsequently, after confirmation of a target, the procedures after the insertion of the spectacle-shaped tube into the C-shaped-cross-section tube of the forceps channel add-on device for an endoscope are repeated. Thus, the forceps can be repeatedly used without additional maneuver of the main body of the endoscope.

Irrespective of whether the external forceps channel device for an endoscope of the present invention is the external forceps channel device for an endoscope to be annexed to the outer periphery of the insertion portion of the existing endoscope or it is the external forceps channel device for an endoscope to be provided on the outer periphery of the insertion portion of the endoscope at the manufacturing stage, the external forceps channel device for an endoscope of the present invention is an invention applicable not only to the colon camera but also to all types of endoscopes for examination and operation, such as a photogastroscope, a bronchoscope, and a cholangiopancreatoscope.

### EFFECTS OF THE INVENTION

The techniques of the present invention individually have any of the following effects.

In the case of providing the forceps channel of the present invention on the outer periphery of the insertion portion of an existing endoscope, if forceps are inserted directly into the C-shaped-cross-section tube, there is a possibility that the forceps do not reach the tip portion of the endoscope that is a target position, protrude on the way of insertion, and may hurt the body of a patient. Accordingly, the external forceps channel connected with the rod guide portion which is sidably insertable into and detachable from the C-shaped-cross-section tube is provided in the C-shaped-cross-section tube. Thus, it is possible to prevent the body of a patient from being hurt and to insert forceps more safely into the C-shaped-cross-section tube than inserting the forceps directly therein.

Moreover, when inserting the external forceps channel device for an endoscope of the type to be added to an existing endoscope, the guide ring in the shape having the protruding center is provided on the tip of the external forceps channel device. Furthermore, the positioning cap formed to match with the shape of the guide ring on the tip is fitted on and fixed to the tip of the endoscope in advance. Thus, when inserting the external forceps channel device, the tip thereof is engaged with the cap on the tip of the insertion portion of the endoscope. As a result, it is possible to prevent an accident that, for example, the external forceps channel device protrudes out of the tip of the insertion portion of the endoscope and thereby hurts the body of a patient, and to perform operations safely. In addition, it is possible to position the external forceps channel device for an endoscope.

The external forceps channel device for an endoscope of the type to be added to an existing endoscope makes it possible to provide a new forceps channel to an existing endoscope easily and at low costs.

The case of providing the forceps channel add-on device for an endoscope on the outer periphery thereof at the manufacturing stage of the endoscope has the following effects.

When inserting the rod guide portion connected with the external forceps channel into either the C-shaped-cross-section groove provided along the surface of the insertion portion of the endoscope in its longitudinal direction or the guide member protruding along the surface of the insertion portion of the endoscope in its longitudinal direction, the linear member having flexibility and elasticity, for example, the guide wire, is passed through the C-shaped-cross-section-groove insertion portion of the rod guide portion in advance. Thus, it is possible to prevent the rod guide portion from being detached from the C-shaped-cross-section groove before the external forceps channel reaches the tip of the endoscope and from damaging a tissue of a patient due to the protrusion, and to move the external forceps channel smoothly up to the tip of the endoscope.

By forming the open portion of the C-shaped-cross-section groove to have the elastic constrictive structure, the connecting portion between the external forceps channel and the rod guide member is pressurized by the material having elasticity. Therefore, in this way as well, it is possible to prevent the rod guide portion connected with the external forceps channel from being detached from the C-shaped-cross-section groove and from damaging a tissue of a patient due to the protrusion similarly to the foregoing case.

As another embodiment of providing the forceps channel add-on device for an endoscope on the outer periphery thereof at the manufacturing stage of the endoscope, the open portion of the C-shaped-cross-section groove is formed to have the elastic constrictive structure. Thus, it is possible to prevent the external forceps channel connected with the rod guide portion slidably inserted into the C-shaped-cross-section groove from being detached and damaging a tissue of a patient due to the protrusion, and to move the external forceps channel smoothly up to the tip of the endoscope.

As still another embodiment of providing the forceps channel add-on device for an endoscope on the outer periphery thereof at the manufacturing stage of the endoscope, the tunnel is provided immediately under the outer peripheral edge of the insertion portion of the endoscope, and magnetic bodies which are freely movable along the surface of the insertion portion of the endoscope in its longitudinal direction are provided inside the tunnel. Moreover, the magnetic bodies are also provided at the bottom portion of the tube constituting the external forceps channel to be inserted along the outside of the insertion portion of the endoscope. Thus, the external forceps channel is insertable and detachable while being guided by the movable magnetic bodies without being detached from the outer periphery of the insertion portion of the endoscope. Accordingly, it is possible to prevent the external forceps channel from being detached from the outer peripheral edge of the insertion portion of the endoscope and from damaging a tissue of a patient due to the projection. Moreover, by providing a shallow groove matching with the shape of the bottom portion of the external forceps channel to insert on a portion above the tunnel on the outer peripheral edge of the insertion portion of the endoscope, the external forceps channel moves smoothly along the outer peripheral surface of the insertion portion of the endoscope.

As yet another embodiment of providing the forceps channel add-on device for an endoscope on the outer periphery thereof at the manufacturing stage of the endoscope, the cross section of the rod guide portion connected with the external forceps channel is formed into a C-shape, and the rail which the C-shaped-cross-section guide portion is engaged with, is insertable into and detachable from, and is smoothly movable along, is provided in the C-shaped-cross-section groove provided on the outer periphery of the forceps channel add-on device for an endoscope. Thus, the guide portion and the external forceps channel are firmly fixed to each other and do not protrude. Accordingly, it is possible to prevent the external forceps channel connected with the rod guide portion from being detached and from damaging a tissue of a patient due to the protrusion, and to move the external forceps channel smoothly up to the tip of the endoscope.

Any of the aspects of the present invention enables the use of two forceps or more without restricting the luminous intensity and the field of view of a ubiquitous endoscope by providing the device for adding a new forceps channel to an endoscope.

Since a conventional forceps channel also serves as a suction port during suction, suction capability of the endoscope is reduced when forceps are inserted. Accordingly, the field of view of the endoscope may become worse and the life of a patient and the like may be endangered. By providing the external forceps channel, it is possible to use the conventional forceps channel as the suction port or to clean the endoscope without reducing the suction power, to improve the field of view, to perform various treatment operations such as a hemostatic operation easily and safely, and to reduce a burden on the patient caused by medical treatments.

In addition, it is possible to extract a large tissue which could not be evulsed through a forceps channel provided in the endoscope of the prior art, by extracting the tissue together with the external forceps channel in a state where the tissue is grasped with forceps inserted through the external forceps channel and in the state where the insertion portion of the endoscope is not drawn out. The forceps can be inserted again, drawn out and inserted repeatedly, and thus it is possible to evulse a plurality of tissues and foreign substances without maneuvering the main body of the endoscope.

For example, in the case of bleeding as a result of resecting an affected part by use of forceps, there are some occasions when a CCD camera on the tip of the endoscope may be tainted by blood due to the bleeding and observation of the affected part may become difficult. However, since the forceps channel also serves as the suction port during suction, suction capability of the endoscope is reduced when the forceps are inserted. Accordingly, because of deterioration in the field of view of the endoscope due to the bleeding or the like, and reduction in the suction power attributable to the insertion of the forceps, the life of a patient and the like may be endangered.

However, by using the external forceps channel device for an endoscope of the present invention, it is possible to use the forceps channel of the conventional endoscope as the suction port and to pour water and the like efficiently from the forceps channel to clean the CCD camera of the endoscope. Thus, it is possible to improve the field of view of the CCD camera and to perform a hemostatic treatment and the like efficiently.

In addition, by using the external forceps channel device for an endoscope provided on the outer periphery of the insertion potion of the endoscope, it is possible to evulse a tissue or foreign substance larger than the bore diameter of the forceps channel incorporated in the endoscope.

For example, basket forceps which are one type of useful forceps can securely grasp a large tissue or foreign substance, and are therefore very effective forceps for removal. However, in many cases of the conventional endoscopes, the bore diameter of the forceps channel incorporated in the endoscope is smaller than the size of a tissue or foreign substance grasped by the basket forceps. Accordingly, in such a case, it was necessary to draw the whole endoscope out. When there were a plurality of large tissues and foreign substances, it was necessary to insert the main body of the endoscope many times. However, such operation was almost impossible in terms of burdens on a patient and an operator, and the use of the endoscope had limitations. However, the use of the external forceps channel device for an endoscope of the present invention makes it possible to remove a plurality of tissues and foreign substances which are tissues to be resected larger than the bore diameter of the forceps channel of the endoscope, by repeating insertion and extraction using the above-described external forceps channel in the state where the insertion portion of the endoscope is not drawn out, without imposing a burden a the patient. Accordingly, it is possible to achieve effective utilization of the present invention.

Moreover, since it is possible to provide a plurality of forceps channels, various treatments can be carried out which have been physically and technically difficult to perform with a pair of forceps. For example, a medical treatment of burning off a tumor or the like with forceps used as a high-frequency snare and extracting the resected tissue through the forceps channel of the external forceps channel device for endoscope can be performed relatively easily.

As described above, resection and evulsion of tissues, foreign substances and the like are facilitated, and leftovers after the treatment are decreased. Moreover, it is possible to evulse a substance larger than the bore diameter of a forceps channel incorporated in an endoscope without drawing out the endoscope itself, by extracting the substance together with the whole external forceps channel while being guided by the guide along the outside of the endoscope, in the state where the insertion portion of the endoscope is not drawn out and where the substance is grasped with the forceps inserted through the external forceps. Accordingly, extremely large burdens on a patient and an operator are substantially relieved.

At the same time, it is possible to secure safety after the treatment such as resection and to contribute to prevention of a complication attributable to the endoscope.

Moreover, since a resected tissue can be extracted without drawing the endoscope out, it is also possible to carry on endoscopy subsequently after an operation.

Therefore, improvement in regional diagnostic capability for a lesion by endoscopy is expected.

1. An external forceps channel device for an endoscope, provided with an external forceps channel which is capable of being repeatedly inserted and extracted in a way of being guided by a guide provided on an endoscope separately and independently therefrom along an outside of an insertion portion of the endoscope while using the endoscope without drawing it out, the endoscope incorporating an air supply path, a light source, a CCD camera, and a forceps channel and including the insertion portion and an maneuvering portion, characterized in that provided is the external forceps channel capable of repeatedly extracting a foreign substance larger than a bore diameter of the incorporated forceps channel in a way of being guided by the guide along the outside of the endoscope, together with the whole external forceps channel itself in a state where the foreign substance is grasped by forceps inserted through the external forceps channel, and that provided is the external forceps channel capable of being repeatedly inserted in a way of being guided by the guide along the outside of the endoscope in a state where the endoscope is not drawn out.

2. The external forceps channel device for an endoscope according to claim 1,
wherein the external forceps channel device for an endoscope is an external forceps channel device capable of being slidably fitted along the outside of the insertion portion of an existing endoscope, and
wherein detachable notched ring-shaped fitting pieces are provided on an outer peripheral surface of the insertion portion at given intervals; a linear member having flexibility and elasticity is fixed to one end side of the fitting pieces to interconnect the fitting pieces; a tube having flexibility with a cross-section of a C-shape is fixed to the other end side of the fitting pieces in parallel with the linear member to interconnect the fitting pieces; and the external forceps channel connected with a rod guide portion that is slidably insertable into and detachable from the C-shaped-cross-section tube is provided in the C-shaped-cross-section tube.

3. The external forceps channel device for an endoscope according to claim 2,
wherein the linear member is any of a tension coil spring with no hook on either end and a rod member made of thermoplastic resin having flexibility and elasticity such as a nylon material.

4. The external forceps channel device for an endoscope according to claim 2 or 3,
wherein a guide ring fitted on a tip of the external forceps channel device for an endoscope is formed into a shape having a protruded center; a guide portion is formed by cutting the guide ring obliquely from a tip toward a base portion thereof, and a positioning cap formed to match with the shape of the guide portion is fitted on a tip of the insertion portion of the endoscope, and
wherein the guide ring on the tip of the external forceps channel device for an endoscope is positioned by aligning the guide ring on the tip of the device with the positioning cap fitted on the tip of the endoscope, at the tip of the endoscope when the external forceps channel device for an endoscope is inserted.

5. The external forceps channel device for an endoscope according to claim 1,
wherein the external forceps channel for the endoscope is provided either in such a manner that a groove having a cross-section of a C-shape is provided along a surface of the insertion portion of the endoscope in its longitudinal direction, and the external forceps channel connected with a rod guide portion which is insertable into and detachable from the C-shaped-cross-section groove while sliding therealong, is provided, or in such a manner that a protruding guide member is provided along the surface of the insertion portion of the endoscope in its longitudinal direction, and the external forceps channel connected with the rod guide portion which is insertable into and detachable from the guide member while being guided by the guide member, is provided.

6. The external forceps channel device for an endoscope according to claim 1 or 5,
wherein the external forceps channel for the endoscope is an external forceps channel one side of which is connected with the rod guide portion that is insertable into any of the C-shaped-cross-section groove and the protruding guide member and sidably insertable into and detachable from the cross-section of any of the C-shaped-cross-section groove and the protruding guide member, and
wherein a guide wire as a core is provided in a central portion of the rod guide portion.

7. The external forceps channel device for an endoscope according to claim 5 or 6,
wherein, for the external forceps channel for the endoscope, an open portion of any of the C-shaped-cross-section groove and the protruding guide member is formed to have an elastic constrictive structure to prevent the external forceps channel from protruding out, the external forceps channel being connected with the rod guide portion sidably inserted into any of the C-shaped-cross-section groove and the protruding guide member.

8. The external forceps channel device for an endoscope according to claim 1,
wherein the external forceps channel device for an endoscope adopts a structure in which a tunnel is provided immediately under a surface of the insertion portion of the endoscope along the surface in its longitudinal direction; a freely movable magnetic body is provided in the tunnel; another magnetic body is also provided at a bottom portion of a tube through which forceps are inserted along the outside of the insertion portion of the endoscope; and when inserting the external forceps channel device, the device is inserted while being guided by the movable magnetic body.

9. The external forceps channel device for an endoscope according to claim 5 or 6,
wherein, for the external forceps channel for the endoscope, a cross-section of the guide portion connected with the external forceps channel is formed into a C-shape, and a rail is provided in any of the C-shaped-cross-section groove and the protruding guide member which are to be engaged with the C-shaped-cross-section guide portion.
